(19) United States
(12) Patent Application Publication
(10) Pub. No.: US 2026/0200833 A1
(43) Pub. Date: Jul. 16, 2026

(54) PRODUCTION OF PANTHENOL

(71) Applicant: DSM IP Assets B.V., Maastricht (NL)

(72) Inventors: René Tobias STEMMLER, Kaiseraugst (CH); Jonathan Alan MEDLOCK, Kaiseraugst (CH); Stefan Karl LAEUGER, Kaiseraugst (CH); Peter Louis HOUSTON, Kaiseraugst (CH)

(21) Appl. No.: 19/130,933

(22) PCT Filed: Nov. 15, 2023
(86) PCT No.: PCT/EP2023/081898

§ 371 Date: May 16, 2025

Related U.S. Application Data
(60) Provisional application No. 63/426,976, filed on Nov. 21, 2022.

(30) Foreign Application Priority Data
Dec. 1, 2022 (EP) 22210854.0

(51) Int. Cl. C07C 231/12; C07C 235/08; C12P 13/02

(52) U.S. Cl. CPC C07C 231/12; C07C 235/08; C12P 13/02

(57) ABSTRACT

The present invention provides a novel process for producing panthenol from pantothenic acid or salts and/or esters thereof. The process makes a “hybrid process” possible to produce panthenol, which is generally more eco-friendly and sustainable and reduces carbon print compared to a pure chemical process.

The present invention relates to a novel process for producing panthenol from pantothenic acid or salts and/or esters thereof.

Panthenol is an alcohol derivative of pantothenic acid or vitamin B₅ which is commercially used in various cosmetic products. Conventional production of panthenol is through chemical synthesis by condensing 3-aminopropanol with pantolactone, which is produced in industry by several chemical steps and normally needs an additional resolution treatment to obtain the desired (R)-pantolactone.

Over the years, several microbial fermentation processes were developed aiming to replace the existing chemical processes. Nowadays use of the genetically engineered strain Bacillus subtilis represents the most efficient process for producing pantothenic acid and salts thereof by fermentation. (see WO0121772 and WO02057474) However, there is still no report on a fermentation processes for producing panthenol directly.

Accordingly, there is an ongoing demand in industry to effectively convert fermentatively produced pantothenic acid into panthenol.

Hence, the present invention provides a new process for producing panthenol from pantothenic acid or salts and/or esters thereof, said process comprising the conversion of pantothenic acid, or salts and/or esters thereof, particularly fermentatively produced pantothenic acid or salts thereof, into panthenol. As said process can use biotechnologically produced compounds including but not limited to pantothenic acid or salts and/or esters thereof, said route furthermore can satisfy the need for production of eco-friendly, sustainable and carbon neutral products.

Particularly, the present invention provides a process for producing a compound of formula (I), comprising the step of reacting a compound of formula (II) with an amino alcohol of formula (III) to obtain the compound of formula (I)

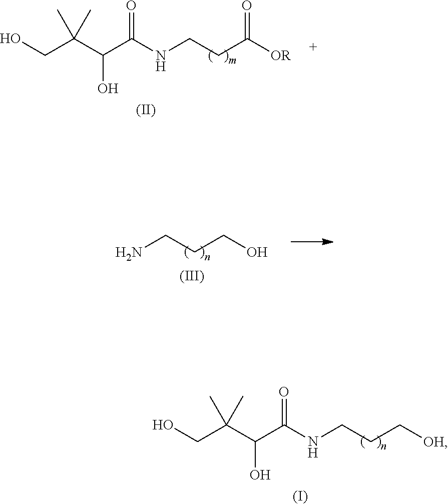

wherein R is selected from the group consisting of M or hydrogen or substituted or unsubstituted alkyl, wherein M is an alkali metal or earth alkali metal, particularly sodium, potassium, calcium, or magnesium, and each of m and n is independently an integer between 0-5.

Preferably, the substituted or unsubstituted alkyl is selected from substituted or unsubstituted C₁-C₁₀ alkyl, such as e.g. C₁, C₂, C₃, C₄, C₅, C₆, C₇, C₈, C₉ or C₁₀-alkyl, more preferably selected from methyl, ethyl, n-propyl, iso-propyl, n-butyl, iso-butyl, tert-butyl, or benzyl.

Preferably, each of m and n is independently 1, 2 or 3, and more preferably, m and n are independently 1.

In the present invention, the compound of formula (II) is preferably selected from pantothenic acid (R=H), a salt of pantothenic acid (M is an alkali metal or earth alkali metal), such as calcium-pantothenate, sodium-pantothenate, magnesium-pantothenate and potassium-pantothenate or pantothenic acid esters (R=substituted or unsubstituted alkyl) including but not limited to pantothenic acid methyl ester or pantothenic acid ethyl ester, wherein these compounds can either be produced by a chemical process as e.g. described in Martin et al. (J. Am. Chem. Soc., Vol. 116, No. 11, 1994) or in WO2017099822, or by a biocatalytic process or preferably via a fermentative process as e.g. described in WO0121772 or WO02057474, including esterification of fermentatively produced pantothenic acid.

It is well understood that in case of M being earth alkali metal, the respective ions are divalent and, hence, formally such metals in the formula (II) would be ½, e.g. ½ Ca, ½ Mg.

In the present invention, the compound of formula (II) is preferably pantothenic acid or a salt thereof, more preferably pantothenic acid or the calcium salt thereof.

In the present invention, the compound of formula (II) is preferably fermentatively produced.

In the present invention, the amino alcohol of formula (III) is preferably 3-aminopropanol.

In the present invention, the compound of formula (I) is preferably panthenol.

In the present invention, the compound of formula (I) or the compound of formula (II) may be present in any configuration, such as e.g. in the (R) or (S)-configuration or occurring as racemate in (R/S)-configuration, or an enantiomerically enriched mixture where one isomer is in excess, whereby the (R) or (R/S)-configuration is preferred. Typically, if the compound of formula (II) is produced by a fermentation process, a percentage of at least about 95%, such as e.g. about 97, 98, 99 or even 100% of (R)-configuration based on total weight of the compound of formula (II) is given.

In the process of the present invention, the amino alcohol of formula (III) may be used in an amount of from 1 mole to 20 moles, preferably from 3 moles to 15 moles, more preferably from 5 moles to 10 moles, per 1 mole of the compound of formula (II).

In the process of the present invention, the reaction advantageously is carried out in the presence of a catalyst which may be a protic acid and/or a Lewis acid.

The protic acid suitable for the process of the present invention may be any organic acid such as carboxylic acids (for example, formic acid, acetic acid, benzoic acid and proline), and/or any inorganic acid such as sulfonic acid, sulfuric acid, hydrochloric acid, phosphoric acid, hydroxylamine-hydrochloride, boric acid (B(OH)₃) and phenylboronic acid (PhB(OH)₂).

The Lewis acid suitable for the process of the present invention may be any metal salt, such as salts of aluminum (AI), bismuth (Bi) and transition metals (for example, zinc (Zn), copper (Cu), scandium (Sc) and lanthanide such as lanthanum (La), europium (Eu), neodymium (Nd) and ytterbium (Yb)), of triflate, sulfonate, acetate, and halide; and any metal amide. Examples of the Lewis acid include but are not limited to Sc(OTf)₃, La(OTf)₃, Eu(OTf)₃, Nd(OTf)₃, Yb(OTf)₃, Eu(OTf)₃, Cu(OAc)₂, Ti(NMe₂)₄ and Al(NMe₂)₃.

Preferably, the catalyst used in the process of the present invention is an inorganic acid such as B(OH)₃ and/or a metal triflate such as a transition metal triflate. More preferably, the catalyst is B(OH)₃ and/or Sc(OTf)₃.

In the process of the present invention, the catalyst may be used in an amount of from 0.0001 moles to 1 mole, preferably from 0.001 moles to 0.1 moles, more preferably from 0.005 moles to 0.05 moles, per 1 mole of the compound of formula (II).

In the process of the present invention, an additional solvent may be used but it is not preferable. The solvent may be a non-aqueous, organic, polar or non-polar solvent. Suitable solvents may be selected from alcohols and polyols, esters, ethers, amides, nitriles, hydrocarbons or chlorinated hydrocarbons with or without substitutions. Preferably, no additional solvent is used in the process of the present invention.

In the process of the present invention, the reaction may be carried out at elevated temperatures. The term “elevated temperature” as used herein includes but is not limited to temperatures in the range of about 40° C. to about 150° C., preferably of about 60° C. to about 120° C. such as 80° C., wherein panthenol is produced.

Preferably, the present invention provides a process for producing panthenol, comprising the step i) of fermentative production of pantothenic acid/pantothenate optionally followed by esterification thereof into pantothenic acid esters of formula (II), and the step ii) of reacting the pantothenic acid or salts and/or esters thereof obtained in the step i) with 3-aminopropanol to obtain panthenol, wherein the step ii) is carried out as described above according to the present invention.

Fermentative production of pantothenic acid is known in the art, see e.g. WO0121772 or WO02057474. The esterification can be done according to standard procedures in the art, including but not limited to biocatalytic or fermentative processes. Chemical esterification, e.g. by reacting pantothenic acid with the respective alcohol in the presence of an acid is however preferred. As used herein, such process combining chemical production steps and biotechnological steps is referred to as a “hybrid process”.

Even more preferably, in all embodiments of the present invention, the use of pantothenic acid derived from microorganism metabolism of plant-derived sugars or alcohol composed of carbon of atmospheric origin, and not composed of fossil-fuel carbon is preferred. Said pantothenic acid has an anthropogenic CO₂ emission profile of zero upon biodegradation, which is particularly desired.

The product comprising the compound of formula (I) can be purified (when needed) using commonly known methods, such as e.g. distillation as described in e.g. US20120149903.

The process of the present invention makes a “hybrid process” possible to produce the compound of formula (I) such as panthenol, which is generally more eco-friendly and sustainable and reduces the carbon footprint compared to a pure chemical process using fuel based raw materials. In addition, the process of the present invention provides the compound of formula (I) such as panthenol preferably in (R)-configuration, wherein at least about 95% such as about 97, 98, 99 or even 100% of the compound of formula (I) is present as (R)-configuration based on the total weight of the compound of formula (I).

The invention is illustrated by the following Examples. All percentages in the examples are related to the weight.

### EXAMPLE 1

(R)-Pantothenic acid (1.63 g, 69% purity, 5.11 mmol) was added to a dried 50 mL Schlenk flask. 3-aminopropan-1-ol (3.88 g, 3.95 mL, 51.1 mmol) and Sc(OTf)₃ (127 mg, 0.255 mmol) was added and heated to 80° C. for 4 h. After cooling the obtained crude oil was purified twice by flash chromatography on silica gel (eluent dichloromethane/MeOH/water 75:22.5:2.5) to obtain (R)-panthenol (0.75 g, 92.6% purity, 66% yield) as colorless oil.

### EXAMPLE 2

(R)-Pantothenic acid (3.2 g, 81.7% purity, 12 mmol), boric acid (37 mg, 0.60 mmol) and 3-aminopropan-1-ol (9.0 g, 9.2 mL, 99% purity, 0.12 mol) were weighted into an argon flushed 20 mL vial. The vial was sealed and the mixture was stirred for 16 h at 80° C. The obtained reaction mixture was dissolved in 10 mL dichloromethane/methanol 3:1 (v/v) and filtered over 100 g silica gel, eluting with dichloromethane/methanol 3:1. The fractions were concentrated to give: 1.48 g of fraction 1 containing 61.2% (qNMR) of (R)-panthenol and 3.0 g of fraction 2 containing 36.5% (qNMR) (R)-panthenol. Total yield of (R)-panthenol: 2.0 g (81%).

1. A process for producing a compound of formula (I), comprising the step of reacting a compound of formula (II) with an amino alcohol of formula (III) to obtain the compound of formula (I) 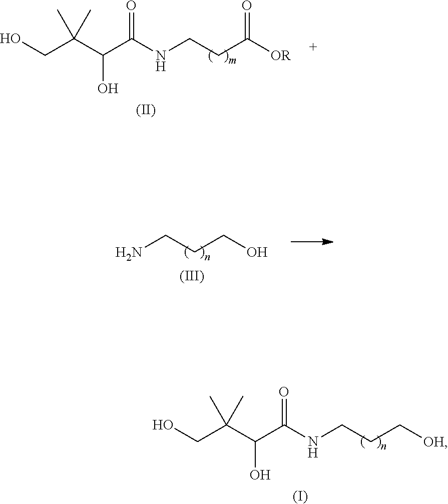
wherein R is selected from the group consisting of M or hydrogen or substituted or unsubstituted alkyl, wherein M is an alkali metal or earth alkali metal, particularly sodium, potassium, calcium, or magnesium, and each of m and n is independently an integer between 0-5.

2. The process of claim 1, wherein the compound of formula (II) is selected from pantothenic acid, a salt of pantothenic acid such as calcium-pantothenate, sodium-pantothenate, magnesium-pantothenate and potassium-pantothenate, or pantothenic acid esters such as pantothenic acid methyl ester or pantothenic acid ethyl ester, preferably pantothenic acid.

3. The process of claim 1, wherein the amino alcohol of formula (III) is 3-aminopropanol.

4. The process of claim 1, wherein the compound of formula (II) is fermentatively produced.

5. The process of claim 1, wherein the amino alcohol of formula (III) is used in an amount of from 1 mole to 20 moles, preferably from 3 moles to 15 moles, more preferably from 5 moles to 10 moles, per 1 mole of the compound of formula (II).

6. The process of claim 1, wherein the reaction is carried out in the presence of a catalyst.

7. The process of claim 6, wherein the catalyst is a protic acid and/or a Lewis acid.

8. The process of claim 7, wherein the protic acid is any organic acid such as carboxylic acids (for example, formic acid, acetic acid, benzoic acid and proline), and/or any inorganic acid such as sulfonic acid, sulfuric acid, hydrochloric acid, phosphoric acid, hydroxylamine-hydrochloride, boric acid (B(OH)₃) and phenylboronic acid (PhB(OH)₂).

9. The process of claim 7, wherein the Lewis acid is any metal salt, such as salts of aluminum (Al), bismuth (Bi) and transition metals (for example, zinc (Zn), copper (Cu), scandium (Sc) and lanthanide such as lanthanum (La), europium (Eu), neodymium (Nd) and ytterbium (Yb)), of triflate, sulfonate, acetate, and halide, and any metal amide, including but not limited to Sc(OTf)₃, La(OTf)₃, Eu(OTf)₃, Nd(OTf)₃, Yb(OTf)₃, Eu(OTf)₃, Cu(OAc)₂, Ti(NMe₂)₄ and Al(NMe₂)₃.

10. The process of claim 6, wherein the catalyst is an inorganic acid such as B(OH)₃ and/or a metal triflate such as a transition metal triflate, preferably the catalyst is B(OH)₃ and/or Sc(OTf)₃.

11. The process of claim 1, wherein no solvent is used in the reaction.

12. The process of claim 1, wherein the reaction is carried out at a temperatures in the range of about 40° C. to about 150° C., preferably of about 60° C. to about 120° C. such as 80° C.

13. The process of claim 1, wherein the compound of formula (I) is so produced that at least about 95% such as about 97, 98, 99 or even 100% of the compound of formula (I) is present as (R)-configuration based on the total weight of the compound of formula (I).

14. A process for producing panthenol, comprising the step i) of fermentative production of pantothenic acid/pantothenate optionally followed by esterification thereof into pantothenic acid esters of formula (II), and the step ii) of reacting the pantothenic acid or salts and/or esters thereof obtained in the step i) with 3-aminopropanol to obtain panthenol.
